(19) United States
(12) Patent Application Publication
(10) Pub. No.: US 2022/0409780 A1
(43) Pub. Date: Dec. 29, 2022

(54) Process for Preparing Blood Components and Biomedical Device

(71) Applicants: MEDITALIA INDUSTRIALE S.R.L., Lovero (Sondrio) (IT); FONDAZIONE POLICLINICO UNIVERSITARIO AGOSTINO GEMELLI IRCCS, Roma (IT); UNIVERSITÀ CATTOLICA DEL SACRO CUORE, Milano (IT); FONDAZIONE IRCCS CA' GRANDA OSPEDALE MAGGIORE POLICLINICO, Milano (IT)

(72) Inventors: Paolo REBULLA, Milano (IT); Giovanni MAZZARO, Lovero, Sondrio (IT); Maria BIANCHI, Milano (IT); Luciana TEOFILI, Milano (IT)

(21) Appl. No.: 17/756,167

(22) PCT Filed: Nov. 18, 2020
(86) PCT No.: PCT/IB2020/060853

§ 371 Date: May 18, 2022

(30) Foreign Application Priority Data
Nov. 18, 2019 (IT) 102019000021426

(51) Int. Cl. A61M 1/02

(52) U.S. Cl. CPC A61M 1/0272; A61M 1/0209; A61M 1/0218; A61M 2202/0427; A61M 2202/0429

(57) ABSTRACT

A process for preparing blood components from blood, by means of a biomedical device (16), comprising the steps of: subjecting an isolated blood sample (1) to a first centrifugation at a speed of 250 rpm for a time of 10 minutes, and to a second centrifugation at a speed of 2000 rpm for a time of 15 minutes.

The present invention relates to a process for preparing blood components and a biomedical device for the production, storage, and traceability of biological products, in particular of blood components.

Modern transfusion therapy techniques are based on the use of blood from adult donors, collected in systems of multiple plastic bags and subjected to centrifugation to separate the main blood components, namely:
red blood cell concentrates, used for treating acute and chronic anemia;
platelet concentrates, used for preventing and treating bleeding;
platelet-poor plasma, used in patients with deficiencies in coagulation factors.

In the prior art, red blood cell concentrates and platelet concentrates are subjected to filtration to eliminate the white blood cells, which can cause reactions and severe side effects in transfused patients.

The preparation of blood components in concentrated form allows patients to be administered only the blood component of which they are lacking in relatively small volumes, optimizing the use of blood collected from donors and avoiding the risk of overloading the patients' cardiovascular system.

Parallel to the consolidated therapeutic use of adult donors' blood, therapeutic protocols have been developed which use placental blood, collected at the end of birth from healthy newborns and donated to the community, for the purpose of transplantation of hematopoietic stem cells in patients with severe hematological diseases.

More than 90% of placental blood donations do not contain a sufficient number of hematopoietic stem cells to perform the transplant safely and are systematically disposed of in hospital waste.

Research projects, procedures for preparing blood components and protocols for the therapeutic use of donated placental blood have been developed as alternatives to hematopoietic transplantation, based on the use of units which are not suitable for such a use.

In particular, systems have been developed which facilitate the preparation of red blood cells, platelet gel and plasma-eye drops of placental blood and the therapeutic administration of platelet gel for treating skin lesions.

A system of this type is disclosed, for example, in patent applications IT102015000020430 and IT102015000020415.

US2016/354280A1 discloses a multiple bag system and a method for preparing blood components.

U.S. Pat. No. 4,596,657A discloses a multiple bag system with integrated filtering means.

US2009/317305A1 discloses a bag system for separating blood in discrete volumes.

US2015/328392 discloses a system and a method for removing white blood cells from a blood sample.

The prior-art procedures for preparing blood components from placental blood have several important limitations.

Firstly, the low-speed centrifugation procedure used to separate platelet-rich plasma from red blood cells does not allow to obtain a high hematocrit value (>60%) in the red blood cells after the re-suspension thereof in a sufficient volume of an adequate additive solution necessary for storing red blood cells for transfusion use. Maintaining a hematocrit value above 60% is a standard requirement for red blood cells for transfusion use.

However, the centrifugation of placental blood must occur at low speed in order to keep the platelets suspended in the plasma fraction which forms in the upper compartment of the bag.

In fact, any high-speed centrifugation of placental blood would cause the collapse of the platelets contained in the plasma, which would prevent a subsequent preparation of a platelet concentrate from the platelet-rich plasma.

A further problem of the prior art is that using red blood cells from placental blood for transfusion purposes requires a medical device adapted to remove leukocytes. The current filtration systems routinely used for preparing leukoreduced red blood cells from adult donors' blood are not adapted to the leukoreduction of red blood cells from placental blood due to the much lower volume of red blood cells in placental blood (approximately ⅕) and high dead space of the filters used for the leukoreduction of red blood cells from adult donor blood, which would cause a significant loss of neonatal red blood cells trapped in the filter at the end of filtration.

Therefore, it is the object of the present invention to provide a process for preparing blood components (red blood cells, platelet concentrate and platelet-poor plasma), and a biomedical device for the production, storage, and traceability of biological products, in particular of blood components, such as to obviate at least some of the problems highlighted in the prior art.

It is a particular object of the present invention to provide a process for preparing a concentrated red blood cell suspension (i.e., a red blood cell concentrate suspended in an adequate additive solution necessary for storing red blood cells) with a high hematocrit value (>60%), and which is thus suitable for transfusion use, in particular for transfusion use in premature newborns.

It is a further particular object of the present invention to provide a more efficient and rapid process for preparing blood components.

It is a further particular object of the present invention to provide an improved biomedical device for the production, storage, and traceability of blood components.

These and other objects are achieved by the process and device described in the appended claims.

Further features and advantages of the present invention will become more apparent from the description of some preferred embodiments thereof, given below by way of non-limiting example with reference to the accompanying drawings, in which:

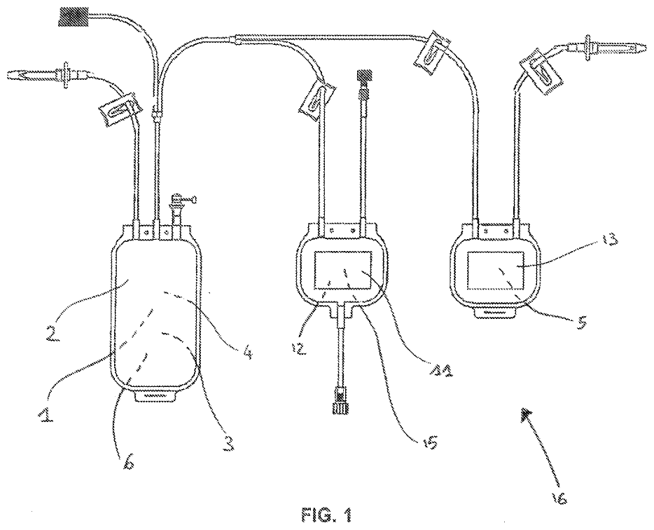

FIG. 1 shows a biomedical device for preparing blood components, according to an embodiment of the invention;

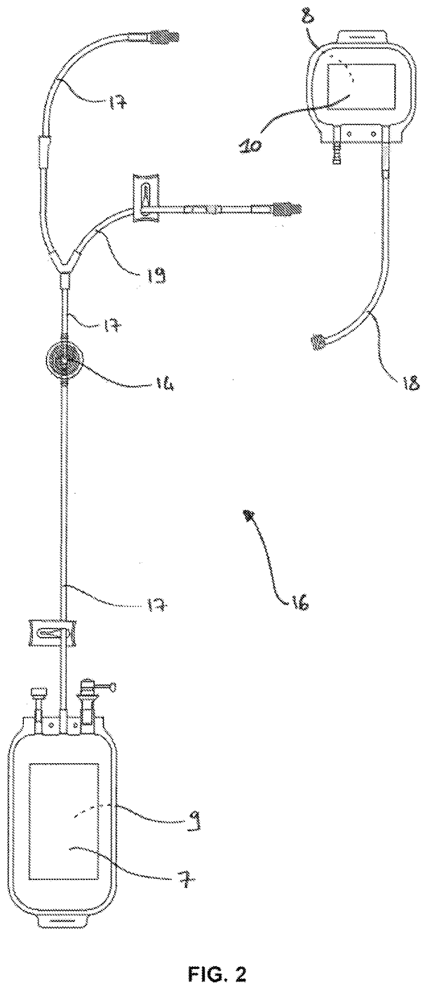

FIG. 2 shows a detail of a biomedical device for filtering red blood cells, according to an embodiment of the invention;

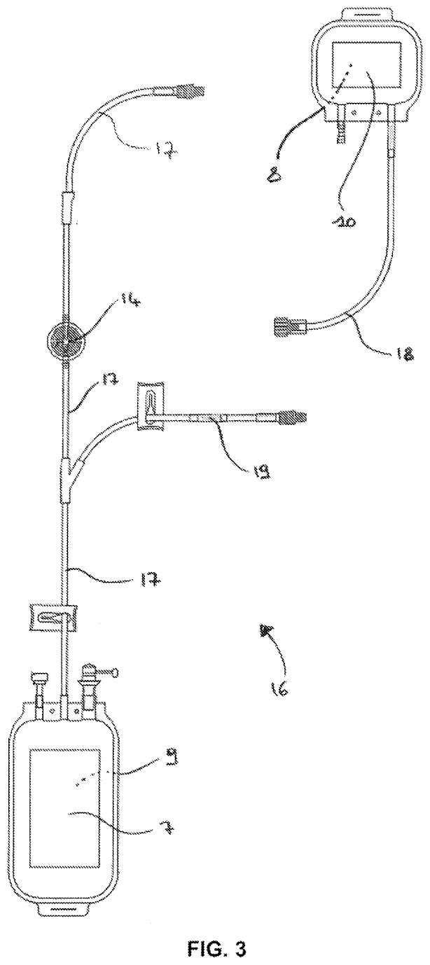

FIG. 3 shows a biomedical device for filtering red blood cells, according to a further embodiment of the invention;

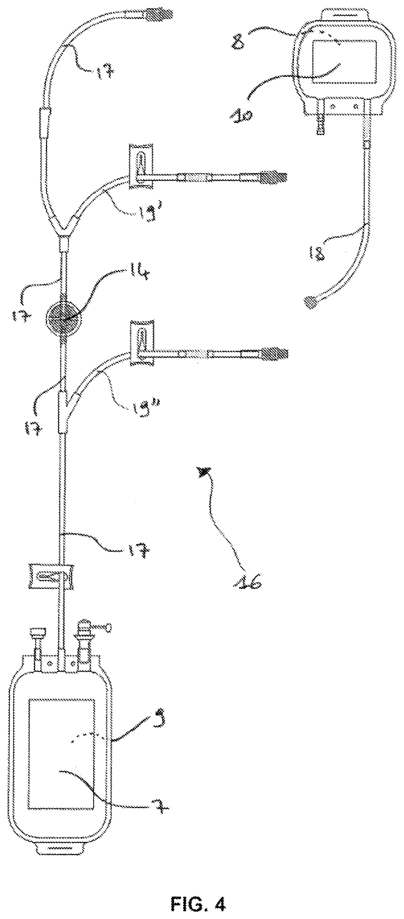

FIG. 4 shows a biomedical device for filtering red blood cells, according to a further embodiment of the invention;

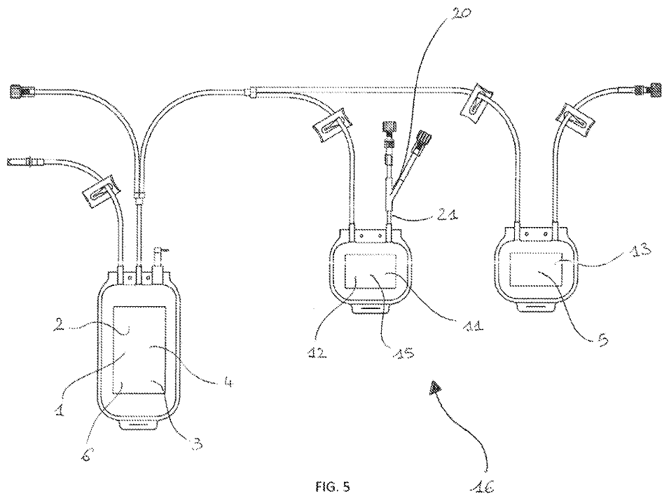

FIG. 5 shows a biomedical device for preparing blood components, according to a further embodiment.

Process for Preparing Blood Components

According to an aspect of the invention, a process for preparing blood components from blood, in particular umbilical cord blood or placental blood, by means of a biomedical device 16 comprising a first bag 2 connected to a second bag 11 and to a third bag 13, comprises the following steps.

A step a) in which an isolated blood sample 1, contained within the first bag 2, is subjected to a first centrifugation at a speed of about 250 rpm for a time of about 10 minutes, so as to obtain a sediment consisting of red blood cells 3 and a supernatant consisting of platelet-rich plasma 4.

Then, a step b) in which the platelet-rich plasma 4 obtained from step a) is transferred from the first bag 2 to the second bag 11.

Then, a step c) in which the first bag 2, connected to the second bag 11 and to the third bag 13, is subjected to a second centrifugation at a speed of 2000 rpm for a time of 15 minutes, so as to obtain a supernatant consisting of platelet-poor plasma 5 in an upper portion of the first bag 2 and a red blood cell concentrate 6 in a lower portion of the first bag 2, and a supernatant consisting of platelet-poor plasma 5 in an upper portion of the second bag 11 and a platelet pad 12 in a lower portion of the second bag 11.

Advantageously, the hematocrit value of the red blood cell concentrate 6 obtained from step c) is over 80%.

With a further advantage, this increases the efficiency of the process since the centrifugation of the first bag 2 and of the second bag 11 occurs simultaneously, inside the same centrifuge.

Then, a step d) in which the platelet-poor plasma 5 is transferred from the first bag 2 to the second bag 11 or to the third bag 13.

Then, a step e) in which the first bag 2 is separated from the second bag 11 and from the third bag 13, and in which the first bag 2 is fluidly connected to a storage bag 7 (FIGS. 2, 3, 4).

Then, a step f) in which the platelet-poor plasma 5 is transferred from the second bag 11 to the third bag 13, minus a volume of platelet-poor plasma 5 such as to dilute the platelet pad 12 forming a re-suspended platelet concentrate 15 having a platelet concentration preferably between 800,000 and 1,200,000 platelets per microliter.

Advantageously, this increases the efficiency of the process since the amount of platelet-poor plasma 5 which can be produced from the original blood sample 1 is increased. In particular, when the first bag 2 has an inner volume of 150 ml, the second bag 11 has an inner volume of 60 ml and the third bag 13 has an inner volume of 60 ml, this process allows to obtain about 10 additional cc of platelet-poor plasma 5.

Then, a step g) in which the red blood cell concentrate 6 is diluted by adding a volume of additive solution 8, so as to obtain a concentrated red blood cell suspension having a hematocrit value over 60%.

Then, a step h) in which the leukocytes contained in the suspension of concentrated red blood cells by filtration are removed through a leukoreduction filter 14 (FIGS. 2, 3, 4), obtaining a filtered concentrated red blood cell suspension 9, and the filtered concentrated red blood cell suspension 9 is collected and stored in the storage bag 7.

Advantageously, a process for preparing blood components thus structured, obtains the preparation of a filtered concentrated red blood cell suspension with a high hematocrit value (>60%), and which is thus suitable for transfusion use, in particular for transfusion use in premature newborns.

With a further advantage, the described process for preparing blood components is efficient and rapid.

With a further advantage, the procedure of the invention specifies the amount of centripetal accelerations (expressed in “rpm”) to which the bags of the system are to be subjected. The procedures known from the prior art do not provide any indication on the accelerations to which the bags (US2016354280, U.S. Pat. No. 4,596,657, US2009317305) or the centrifugation time (U.S. Pat. No. 4,596,657, US2009317305) are to be subjected.

According to one embodiment, the additive solution 8 for storing red blood cells consists of sodium-adenine-glucose-mannitol (SAGM).

Advantageously, SAGM ensures an adequate re-suspension of the red blood cell concentrate 6 and a high storage of the filtered concentrated red blood cell suspension 9 for transfusion use.

Alternatively, the additive solution 8 consists of a physiological solution.

According to an embodiment, the additive solution 8 for storing red blood cells is previously contained in a solution bag 10 which is removably fluidly connectable to the first bag 2.

According to an embodiment, step g) is carried out by fluidly connecting a tributary cannula 18 of the solution bag 10 to a secondary cannula 19, in which the secondary cannula 19 branches off from a main cannula 17 fluidly connecting the first bag 2 to the storage bag 7, by a sterile connection.

According to an embodiment, before carrying out the step a) described above, the process includes a selection step a1), in which a blood unit, in particular placental blood (also called umbilical cord blood), is selected according to the following parameters:
TNC (total nucleated cells)<1.5×10⁹;
Volume without anticoagulant >50 ml;
Platelet count in units with anticoagulant >150000 per microliter;
Beginning of the process within 48 hours from the blood unit collection;
Optionally, compliance with local regulations.

According to an embodiment, following the selection step a1), and before carrying out step a), the process includes a recording step a2), in which the recording of the date and time of the delivery of the blood unit to be allocated to step a), and the date and time of the beginning of step a) is carried out.

According to an embodiment, after step a2) and before step a), the process includes an analysis step a3), in which the net weight of the blood unit is determined and a complete blood count of the blood unit is performed, which is a complete examination of the parameters of the blood contained in the blood unit.

Following step a3) and before step a), the process includes a transfer step a4), in which the blood of the blood unit is transferred by a sterile connection into the first bag 2.

According to an embodiment, in step b), the platelet-rich plasma 4 is extracted from the first bag 2 and transferred to the second bag 11 by a plasma extractor. In one aspect of the invention, this extractor can be of the manual type; alternatively, an automatic extractor can be used.

According to an embodiment, the platelet-poor plasma 5 is extracted from the second bag 11 and transferred to the third bag 13 by a manual plasma extractor, and by a subsequent transfer from the second bag 11 to the third bag through a siphon racking. “Siphon racking” means a transfer of fluids carried out by utilizing the operating principle of the siphon.

Advantageously, the subsequent transfer by siphon racking substantially removes all the platelet-poor plasma from the second bag 11, so that only the platelet pad 12 remains in the second bag 11.

The procedure for siphoning platelet-poor plasma forms one of the main innovations introduced by the procedure of the invention; in fact, no other procedure known from the prior art describes or suggests siphoning the platelet-poor plasma.

In fact, an extraction of platelet-poor plasma 5 performed only by a manual plasma extractor would not obtain a total extraction due to the thickness of the second bag 11.

Following the transfer of the platelet-poor plasma 5 from the second bag 11 to the third bag 13, the net weight of the platelet pad 12 contained in the second bag 11 is calculated.

Following step d), the first bag 2 containing the red blood cell concentrate 6 is sealed, the net weight thereof is determined (therefore the weight of only the red blood cell concentrate 6), a blood count on the red blood cell concentrate 6 contained in the first bag 2 is performed, and the bag 2 is cooled and stored at a temperature between 2° C. and 6° C.

According to one embodiment, the volume of platelet-poor plasma 5 adapted to form a re-suspended platelet concentrate 15 of predetermined concentration of platelets per microliter is calculated by multiplying the total number (in billions) of platelets in the starting blood unit, by the average percentage of platelets recovered in the platelet-rich plasma 4 separated in step b) (determined during the procedure validation protocol).

For example, if the total number of platelets in the starting blood unit is 15 billion, and if the average percentage of recovered platelets is 50%, then the final volume of the re-suspended platelet concentrate 15 having a concentration of approximately 1 million platelets per microliter, is equal to 7.5 ml.

Therefore, for example, if the final volume of the re-suspended platelet concentrate is 7.5 ml (thus having a weight of about 7.5 g), and the previously calculated net weight of the platelet pad 12 contained in the second bag 11 is 3 g, then the weight of the platelet-poor plasma 5 to be transferred to the second bag 11 is 4.5 g.

Advantageously, this calculation method is immediate and has a high degree of accuracy.

Preferably, following the formation of the re-suspended platelet concentrate 15, a complete blood count on the re-suspended platelet concentrate 15 contained in the second bag 11, and a blood count on the platelet-poor plasma 5 contained in the third bag 13 are performed.

The second bag 11 containing the re-suspended platelet concentrate 15, and the third bag 13 containing the platelet-poor plasma 5, are then cooled and stored at temperatures below −25° C.

According to a preferred embodiment, the additive solution 8 is added to the red blood cell concentrate 6 by directing the flow of the additive solution 8 in the opposite direction to the flow of the red blood cell concentrate 6 directed towards the storage bag 7.

According to an embodiment, during the transfer of the red blood cell concentrate 6 from the first bag 2 to the storage bag 7, the red blood cell concentrate 6 is filtered by a leukoreduction filter 14.

Advantageously, the filtration of the red blood cell concentrate 6 by the leukoreduction filter 14 is carried out after the addition of the additive solution 8, thus avoiding the risk of hemolysis which can be generated during the filtration of red blood cells with very high hematocrit.

Biomedical Device for Preparing Blood Components

According to a further aspect of the invention, a biomedical device 16 comprises a first bag 2, connected to a second bag 11 and to a third bag 13.

The biomedical device 16 further comprises a storage bag 7 fluidly connected to the first bag 2 by a main cannula 17.

The biomedical device 16 further comprises a solution bag 10 comprising a tributary cannula 18. The solution bag 10 is separated from the main cannula 17 and fluidly connectable to the secondary cannula 19 which branches off from the main cannula 17 by a sterile connection of the tributary cannula 18 to the secondary cannula 19.

The first bag 2 is adapted to contain a blood sample 1 and a red blood cell concentrate 6.

The solution bag 10 is adapted to contain an additive solution 8 for red blood cell storage.

The storage bag 7 is adapted to contain a filtered concentrated red blood cell suspension 9.

Advantageously, a medical device 16 thus configured ensures the preparation of a filtered concentrated red blood cell suspension 9 with a high hematocrit value (>60%), and which is thus suitable for transfusion use, in particular for transfusion use in premature newborns.

Furthermore, the second bag 11 is adapted to contain platelet-rich plasma 4, platelet-poor plasma 5 and a platelet pad 12, and a re-suspended platelet concentrate 15.

Furthermore, the third bag 13 is adapted to contain platelet-poor plasma 5.

According to an embodiment, the biomedical device 16 further comprises a siphon fluidly connecting the second bag 11 to the third bag 13.

According to an embodiment, the second bag 11 comprises a connecting tube 20 connected to the second bag 11 at an upper region of the second bag 11.

According to a preferred embodiment, the second bag 11 comprises a blind tube 21 placed at the upper region of the second bag 11, and the connecting tube 20 is inserted with a “Y” fitting on the blind tube 21 (FIG. 5).

According to an embodiment, the tributary cannula 18 is configured to input the additive solution 8 into the main cannula 17 by directing the flow of the additive solution 8 in the opposite direction to the flow of the red blood cell concentrate 6 directed from the first bag 2 towards the storage bag 7.

According to an embodiment, the biomedical device 16 comprises a leukoreduction filter 14 arranged in the main cannula 17.

The leukoreduction filter 14 is adapted to filter the red blood cell concentrate 6 directed towards the storage bag 7.

According to an advantageous embodiment, the leukoreduction filter 14 is placed upstream, with reference to the flow of the red blood cell concentrate 6 from the first bag 2 to the storage bag 7, of the intersection between the main cannula 17 and the secondary cannula 19.

According to an alternative embodiment, the leukoreduction filter 14 is placed downstream, with reference to the flow of the red blood cell concentrate 6 from the first bag 2 to the storage bag 7, of the intersection between the secondary cannula 19 and the tributary cannula 18 (FIG. 3). This embodiment allows the perfusion of the leukoreduction filter 14 at the end of the filtration procedure, in order to recover residual red blood cells in the dead space of the leukoreduction filter 14.

According to a further alternative embodiment, a first and a second secondary cannula 19′, 19″ branch off from the main cannula 17, and the leukoreduction filter 14 is between the intersection between the main cannula 17 and the first secondary cannula 19′, and between the intersection between the main cannula 17 and the second secondary cannula 19″ (FIG. 4). This embodiment allows to carry out a washing procedure of the filter before filtration through the first secondary cannula 19′ placed in a position above the leukoreduction filter 14, recovering the washing liquid from the second secondary cannula 19″ placed in a position below the leukoreduction filter 14 after the sterile connection of the leukoreduction filter 14 with a collection bag not comprised in the device.

The embodiments described in FIGS. 2, 3 and 4 allow operators to perform different filtration procedures, corresponding to different needs, options and operating specifications, such as recovering the leukocyte fraction trapped in the leukoreduction filter 14 by elution, for example.

Advantageously, these different connection systems of the additive solution make the system of the invention very versatile, allowing the leukoreduction filter 14 to be wetted from below or from above, and finally rinsing it, if desired, in order to recover more red blood cells.

According to an embodiment, the first bag 2, the second bag 11 and the third bag 13 are made of a flexible material resistant to a 2000 rpm centrifugation with a duration of 15 minutes.

According to an embodiment, the first bag 2 has an inner volume of 150 ml, the second bag 11 has an inner volume of 60 ml, the third bag 13 has an inner volume of 60 ml, the solution bag 10 has an inner volume equal to 50 ml, and the storage bag 7 has an inner volume of 150 ml.

According to a further aspect of the invention, a system for preparing blood components comprises a biomedical device 16 as described above, at least one centrifuge and at least one manual plasma extractor.

According to an embodiment, the centrifugations are performed using a protective tubular flexible casing and cylindrical adapters made of solid plastic into which the system of the bags 2, 11 and 13 is inserted before each centrifugation.

Naturally, those skilled in the art will be able to make modifications or adaptations to the present invention, without however departing from the scope of the claims below.

1. A process for preparing blood components from blood, by means of a biomedical device (16) comprising a first bag (2) connected to a second bag (11) and to a third bag (13), said process comprising the steps of:
a) Subjecting an isolated blood sample (1), contained within the first bag (2), to a first centrifugation at a speed of 250 rpm for a time of 10 minutes, so as to obtain a sediment consisting of red blood cells (3) and a supernatant consisting of platelet-rich plasma (4);
b) Transferring from the first bag (2) said platelet-rich plasma (4) obtained from step a) to the second bag (11);
c) Subjecting the first bag (2), connected to the second bag (11) and to the third bag (13), to a second centrifugation at a speed of 2000 rpm for a time of 15 minutes, so as to obtain a supernatant consisting of platelet-poor plasma (5) in an upper portion of the first bag (2) and a red blood cell concentrate (6) in a lower portion of the first bag (2), and a supernatant consisting of platelet-poor plasma (5) in an upper portion of the second bag (11) and a platelet pad (12) in a lower portion of the second bag (11);
d) Transferring the platelet-poor plasma (5) from the first bag (2) to the second bag (11) or to the third bag (13);
e) Separating the first bag (2) from the second bag (11) and from the third bag (13), and fluidly connecting the first bag (2) to a storage bag (7);
f) Transferring the platelet-poor plasma (5) from the second bag (11) to the third bag (13), minus a volume of platelet-poor plasma (5) such as to dilute the platelet pad (12), thus forming a re-suspended platelet concentrate (15) having a platelet concentration between 800,000 and 1,200,000 platelets per microliter;
g) Diluting the red blood cell concentrate (6) by adding a volume of additive solution (8) so as to obtain a suspension of concentrated red blood cells having a hematocrit value over 60%;
h) Removing the leukocytes contained in the suspension of concentrated red blood cells by filtration through a leukoreduction filter (14), thus obtaining a filtered concentrated red blood cell suspension (9), and collecting and storing the filtered concentrated red blood cell suspension (9) in the storage bag (7).

2. A process according to claim 1, wherein the additive solution (8) for storing the red blood cells consists of sodium-adenine-glucose-mannitol (SAGM), or a physiological solution.

3. A process according to claim 1, wherein the additive solution (8), prior to step g), is contained in a solution bag (10) which is removably fluidly connectable to the first bag (2).

4. A process according to claim 3, wherein step g) is carried out by fluidly connecting a tributary cannula (18) of the solution bag (10) to a secondary cannula (19), wherein the secondary cannula (19) branches off from a main cannula (17) fluidly connecting the first bag (2) to the storage bag (7) by a sterile connection.

5. A process according to claim 1, wherein, before step a), a selection step a1) is included, in which a blood unit is selected according to the following parameters:
TNC (total nucleated cells)<1.5×10⁹;
Volume without anticoagulant >50 ml;
Platelet count in units with anticoagulant >150,000 per microliter;
Beginning of the process within 48 hours from the blood unit collection;
optionally, compliance with local regulations.

6. A process according to claim 5, wherein, following the selection step a1) and before step a), a recording step a2) is included, in which the recording of the date and time of the delivery of the blood unit to be allocated to step a), and of the date and time of the beginning of step a), is carried out.

7. A process according to claim 6, wherein following step a2) and before step a), an analysis step a3) is included, in which the net weight of the blood unit is determined and a blood count of the blood unit is performed,
and following step a3), a transfer step a4) is included, in which the blood of the blood unit is transferred by a sterile connection into the first bag (2).

8. A process according to claim 1, wherein in step b) the platelet-rich plasma (4) is extracted from the first bag (2) and transferred to the second bag (11) by a manual plasma extractor.

9. A process according to claim 1, wherein the platelet-poor plasma (5) is extracted from the second bag (11) and transferred to the third bag (13) by a manual plasma extractor, and by a subsequent transfer from the second bag (11) to the third bag (13) through a siphon racking.

10. A process according to claim 1, wherein following the transfer of the platelet-poor plasma (5) from the second bag (11) to the third bag (13), the net weight of the platelet pad (12) contained in the second bag (11) is calculated.

11. A process according to claim 1, wherein following step d), the first bag (2) containing the red blood cell concentrate (6) is sealed, the net weight of the first bag (2) containing the red blood cell concentrate (6) is determined, a blood count on the red blood cell concentrate (6) contained in the first bag (2) is performed, and the first bag (2) containing the red blood cell concentrate (6) is cooled and stored at a temperature between 2° C. and 6° C.

12. A process according to claim 1, wherein the volume of the platelet-poor plasma (5) adapted to form a re-suspended platelet concentrate (15) of predetermined concentration of platelets per microliter is calculated by multiplying the total number of platelets in the starting blood unit by the average percentage of platelets recovered in the platelet-rich plasma (4) separated in step b).

13. A process according to claim 1, wherein following the formation of the re-suspended platelet concentrate (15), a blood count on the re-suspended platelet concentrate (15) contained in the second bag (11) and a blood count on the platelet-poor plasma (5) contained in the third bag (13) are performed,
and the second bag (11) containing the re-suspended platelet concentrate (15) and the third bag (13) containing the platelet-poor plasma (5) are then cooled and stored at temperatures below −25° C.

14. A process according to claim 1, wherein the additive solution (8) is added to the red blood concentrate (6) by directing the flow of the additive solution (8) in the opposite direction to the flow of the red blood cell concentrate (6) directed towards the storage bag (7).

15. A biomedical device (16) for preparing blood components, comprising:
a first bag (2), said first bag (2) being connected to a second bag (11) and to a third bag (13); said second bag (11) being adapted to contain plasma (4) and a platelet pad (12) and a re-suspended platelet concentrate (15); said third bag (13) being adapted to contain platelet-poor plasma (5);
a storage bag (7), fluidly connected to the first bag (2) by a main cannula (17);
a solution bag (10), comprising a tributary cannula (18);
said solution bag (10) being separated from the main cannula (17) and being fluidly connectable to the secondary cannula (19), branching off from the main cannula (17), by means of a sterile connection of the tributary cannula (18) to the secondary cannula (19); wherein the first bag (2) is adapted to contain a blood sample (1) and a red blood cell concentrate (6),
wherein the solution bag (10) is adapted to contain an additive solution (8) for red blood cell storage,
and wherein the storage bag (7) is adapted to contain a filtered concentrated red blood cell suspension (9).

16. A biomedical device (16) according to claim 15, comprising a siphon fluidly connecting the second bag (11) to the third bag (13).

17. A biomedical device (16) according to claim 15, wherein the second bag (11) comprises a connecting tube (20) connected to the second bag (11) at an upper region of the second bag (11).

18. A biomedical device (16) according to claim 17, wherein the second bag (11) comprises a blind tube (21) placed at the upper region of the second bag (11), and the connecting tube (20) is inserted with a “Y” fitting on the blind tube (21).

19. A biomedical device (16) according to claim 15, wherein the tributary cannula (18) is configured to input the additive solution (8) into the main cannula (17) by directing the flow of the additive solution (8) in the opposite direction to the flow of the red blood cell concentrate (6) directed from the first bag (2) towards the storage bag (7).

20. A biomedical device (16) according to claim 15, comprising a leukoreduction filter (14) arranged in the main cannula (17), said leukoreduction filter (14) being adapted to filter the concentrated red blood cell suspension directed towards the storage bag (7).

21. A biomedical device (16) according to claim 20, wherein the leukoreduction filter (14) is placed upstream, with reference to the flow of the concentrated red blood cell suspension from the first bag (2) to the storage bag (7), of the intersection between the main cannula (17) and the secondary cannula (19).

22. A biomedical device (16) according to claim 20, wherein the leukoreduction filter (14) is placed downstream, with reference to the flow of the red blood cell concentrate (6) from the first bag (2) to the storage bag (7), of the intersection between the secondary cannula (19) and the tributary cannula (18).

23. A biomedical device (16) according to claim 20, wherein a first and a second secondary cannula (19′, 19″) branch off from the main cannula (17), and the leukoreduction filter (14) is between the intersection between the main cannula (17) and the first secondary cannula (19′), and between the intersection between the main cannula (17) and the second secondary cannula (19″).

24. A biomedical device (16) according to claim 15, wherein the first bag (2), the second bag (11) and the third bag (13) are made of a flexible material which is resistant to a 2000 rpm centrifugation with a duration of 15 minutes.

25. A biomedical device (16) according to claim 15, wherein:
the first bag (2) has an inner volume of 150 ml,
the second bag (11) has an inner volume of 60 ml,
the third bag (13) has an inner volume of 60 ml,
the solution bag (10) has an inner volume of 50 ml,
the storage bag (7) has an inner volume of 150 ml.

26. A system for preparing blood components, comprising:
a biomedical device (16) according to claim 15,
at least one centrifuge,
at least one plasma extractor.

27. A system according to claim 26, wherein at least one centrifuge comprises a protective tubular flexible casing and cylindrical adapters made of solid plastic.
